(19) United States
(12) Patent Application Publication
(10) Pub. No.: US 2018/0327808 A1
(43) Pub. Date: Nov. 15, 2018

(54) NANODOT AND METHOD FOR DETECTING GLUCOSE CONCENTRATION THEREOF

(71) Applicant: NATIONAL SUN YAT-SEN UNIVERSITY, Kaohsiung City (TW)

(72) Inventors: SHU-CHEN HSIEH, Kaohsiung City (TW); PEI-YING LIN, Kaohsiung City (TW)

(21) Appl. No.: 15/622,592

(22) Filed: Jun. 14, 2017

(30) Foreign Application Priority Data
May 9, 2017 (TW) 106115335

(51) Int. Cl. C12Q 1/26; C12N 11/02; G01N 21/64

(52) U.S. Cl. CPC C12Q 1/26; C12N 11/02; C12Y 101/03004; G01N 2021/6432; G01N 2400/00; G01N 2610/00; G01N 21/6428

(57) ABSTRACT

A nanodot for detecting glucose concentration includes a silicon oxide core, a self-assembled monolayer having a 3-glycidoxypropyl trimethoxysilane group, and a glucose oxidase particle. The self-assembled monolayer joins the silicon oxide core by a covalent bond, and the glucose oxidase particle joins the 3-glycidoxypropyl trimethoxysilane group of the self-assembled monolayer by a conjugated bond. Moreover, a method for detecting glucose concentration by the nanodot includes oxidizing a glucose molecule in a glucose solution by the glucose oxidase particle of the nanodot, producing a hydrogen peroxide molecule; fluorescent quenching the nanodot by the hydrogen peroxide molecule, resulting in a change in fluorescent intensity of the nanodot; and detecting the change in fluorescent intensity of the nanodot.

### CROSS REFERENCE TO RELATED APPLICATIONS

The application claims the benefit of Taiwan application serial No. 106115335, filed on May 9, 2017, and the entire contents of which are incorporated herein by reference.

### BACKGROUND OF THE INVENTION

### 1. Field of the Invention

The present invention relates to a nanodot and, more particularly, to a nanodot used for detecting glucose concentration. The present invention also related to a method for detecting glucose concentration by using the nanodot.

### 2. Description of the Related Art

Quantum dots (QD) are semiconductor particles with only several nanometers in size. Based on their excellent optical properties, QDs are usually adapted to fluorescent probes for biological analysis. As an example, CdSe/ZnS QDs, assembled with the glucose oxidase (GOx) particles, can be used to monitor glucose concentration of a glucose solution based on their fluorescence quenching by the H₂O₂ molecules produced by the GOx particles.

However, the GOx particles easily lose their enzymatic activity if the GOx particles are immobilized on the CdSe/ZnS QDs. As a result, sensitivity and specificity of the CdSe/ZnS QDs on detecting glucose concentration decrease. In light of this, it is necessary to develop a nanodot for detecting glucose concentration.

### SUMMARY OF THE INVENTION

It is therefore an objective of the present invention to provide a nanodot which can be used to detect glucose concentration sensitively and specifically.

It is another objective of the present invention to provide a method for detecting glucose concentration using the nanodot.

In an aspect, a nanodot for detecting glucose concentration includes a silicon oxide core, a self-assembled monolayer having a 3-glycidoxypropyl trimethoxysilane group, and a glucose oxidase particle. The self-assembled monolayer joins the silicon oxide core by a covalent bond, and the glucose oxidase particle joins the 3-glycidoxypropyl trimethoxysilane group of the self-assembled monolayer by a conjugated bond.

In another aspect, a method for detecting glucose concentration includes oxidizing a glucose molecule in a glucose solution by the glucose oxidase particle of the nanodot, providing a hydrogen peroxide molecule; fluorescent quenching the nanodot by the hydrogen peroxide molecule, resulting in a change in fluorescent intensity of the nanodot; and detecting the change in fluorescent intensity of the nanodot.

In an example, the change in fluorescent intensity of the nanodot is detect at 497 nm.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is a schematic diagram illustrating of the nanodot according to an embodiment of the present invention.

FIG. 2a is an AFM particle size distribution histogram of the self-assembled layer joining the silicon oxide core by the covalent bond.

FIG. 2b is a TEM image of the self-assembled layer joining the silicon oxide core by the covalent bond.

FIG. 3a is a SERS spectrum of the nanodot according to an embodiment of the present invention.

FIG. 3b is a PL spectrum of the nanodot according to an embodiment of the present invention.

FIG. 4 is a PL spectrum illustrating the fluorescent intensity of the nanodot according to an embodiment of the present invention after glucose treatment.

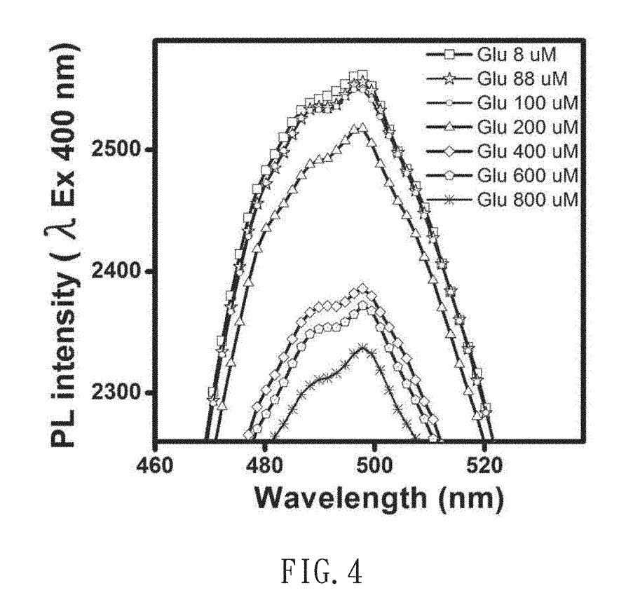

### DETAILED DESCRIPTION OF THE INVENTION

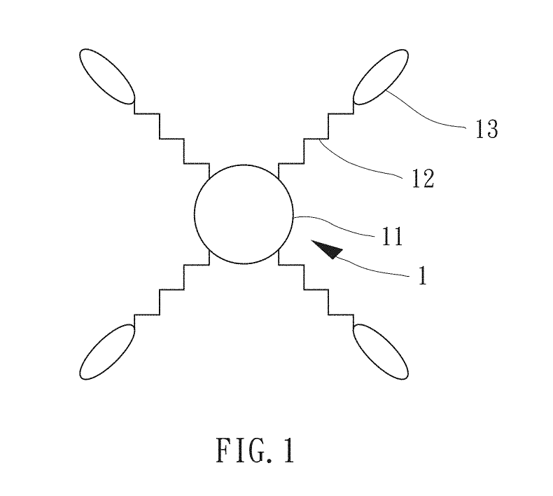

Referring to FIG. 1, a nanodot 1 according to an embodiment of the present invention approximately includes: a silicon oxide (SiOₓ) core 11, a self-assembled monolayer (SAM) 12 and a glucose oxidase particle 13.

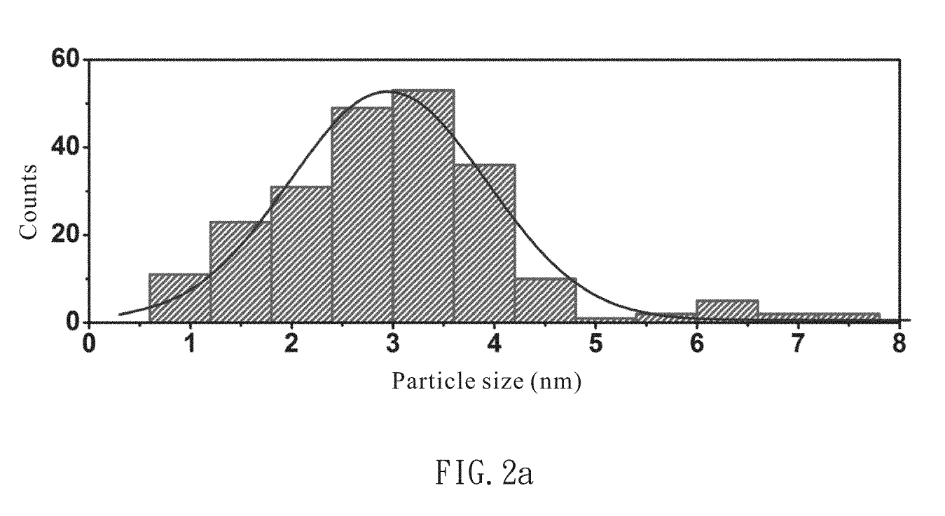

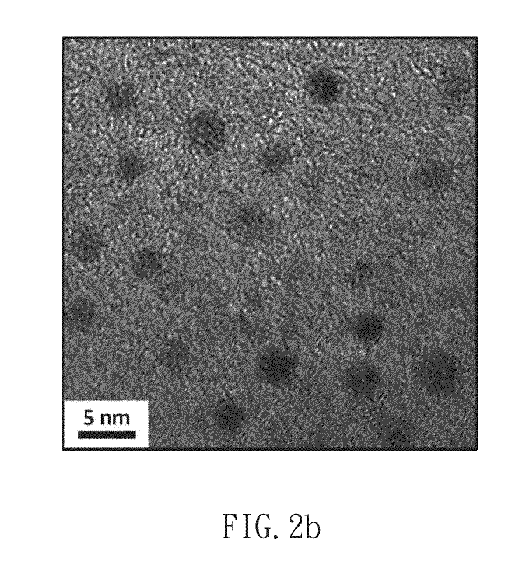

Specifically, 3-glycidoxypropyl trimethoxysilane is hydrolyzed and condensed to form the silicon oxide core 11 and the self-assembled monolayer 12. The self-assembled monolayer 12 can contain a 3-glycidoxypropyl group, and the self-assembled monolayer 12 joins the silicon oxide core 11 by a covalent bond. In this embodiment, 3-glycidoxypropyl trimethoxysilane is hydrolyzed and condensed at 350° C. for 90 minutes under an ambient air atmosphere. After cooling to room temperature, the self-assembled monolayer 12 joining the silicon oxide core 11 by the covalent bond is obtained. For easily understanding, the self-assembled monolayer 12 joining the silicon oxide core 11 by the covalent bond is named as GPS-SAND. Moreover, with reference to FIGS. 2a and 2b, the GPS-SAND has a particle size of 3.1±0.3 nm.

Referring to FIG. 1, an amino group of the glucose oxidase particle 13 can join the 3-glycidoxypropyl group of the self-assembled monolayer 12 via an aminolysis reaction. Thus, the glucose oxidase particle 13 can be stably immobilized on the self-assembled monolayer 12. Moreover, the glucose oxidase particle 13 immobilized on the self-assembled monolayer 12 still shows enzymatic activity because the GPS-SAND has a pH value about 7 (the glucose oxidase particle 13 shows enzymatic activity only in the environment with pH value of 4-7.5). Moreover, no EDC/NHS activator is needed when the glucose oxidase particle 13 is immobilized on the self-assembled monolayer 12, thereby preventing the glucose oxidase particle 13 from depletion due to the complex manufacturing process. In this embodiment, the glucose oxidase particle 13 (2 mg) is dissolved in a phosphate buffer solution (10 μL), followed by being mixed with the GPS-SAND (590 μL). The obtained mixture is sonicated for 30 minutes to form the nanodot 1. All the process is performed under 20-30° C.

The obtained nanodot 1 has a maximum fluorescence emission (λₑₘ) of 497 nm at excitation wavelength (λₑₓ) of 400 nm. The nanodot 1 is therefore able to be applied to detection of glucose concentration in a glucose solution, which is dwelled on as follows.

In the use of detecting glucose concentration, the nanodot 1 according to the embodiment of the present invention is added in the glucose solution. The glucose molecule in the glucose solution is oxidized by the glucose oxidase particle 13 of the nanodot 1, and hydrogen peroxide (H₂O₂) molecule is therefore formed. Then H₂O₂ molecule fluorescent quenches the nanodot, resulting in a change in fluorescent intensity of the nanodot 1. That is, a worker can calculate the glucose concentration of the glucose solution via the change in fluorescent intensity of the nanodot 1.

To validate that the nanodot 1 according to the embodiment of the present invention can be applied to detection of glucose concentration, the following trials are carried out.

Trial (A).

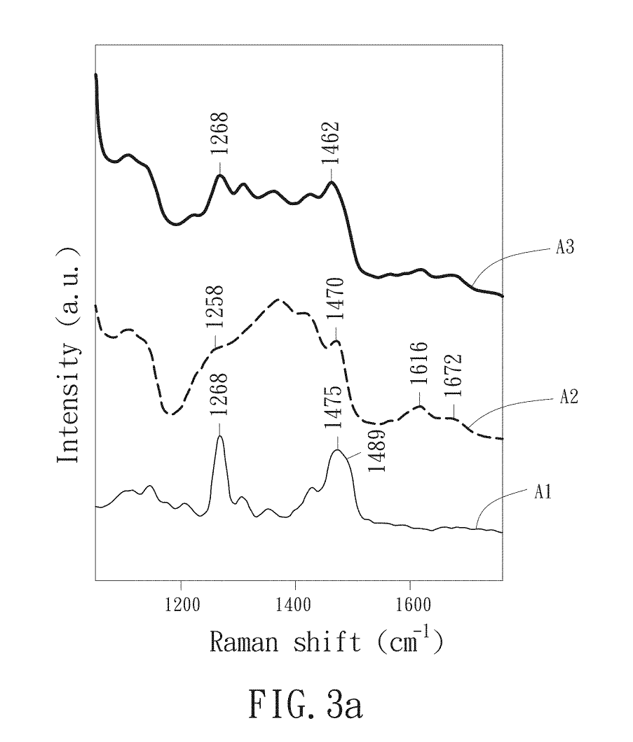

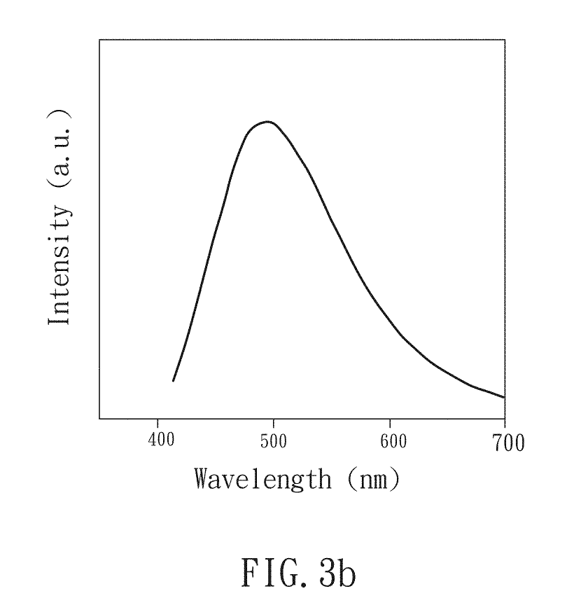

The nanodot 1 is analyzed using SERS (surface-enhanced Raman scattering spectrum), and the SERS spectra are shown in FIG. 3a. The peaks at 1268, 1475 and 1489 cm⁻¹ observed in GPS-SAND (group A1) are characteristics of epoxide ring vibrations. The peaks at 1258, 1672 and 1470 cm⁻¹ observed in the glucose oxidase particle 3 (group A2) are assigned to amide III, amide I and CH₂/CH₃ deformation, respectively. However, the peaks at 1268, 1475 and 1489 cm¹ disappear in the nanodot 1 (group A3) formed by the GPS-SAND and the glucose oxidase particle 3 via the aminolysis reaction.

Moreover, the nanodot 1 is analyzed using PL spectrum (photoluminescence spectrum) at the excitation wavelength of 400 nm. FIG. 2b shows the emission spectra recorded from 400 nm to 700 nm, indicating the nanodot 1 has the maximum fluorescent intensity at 497 nm.

Trial (B).

The nanodot 1 is mixed with 1 μL of glucose at various different concentrations ranging from 8 to 800 μM. The mixture is sonicated for 20 minutes at room temperature, followed by being analyzed by PL spectrum. With reference to FIG. 3, the maximum fluorescent intensity at 497 nm of the nanodot 1 decreases as the glucose concentration increases. Moreover, the change in fluorescent intensity at 497 nm is linearly correlated with the glucose concentration of the glucose solution, which varies from 88 μM to 400 μM (R²=0.99).

Accordingly, the nanodot according to the present invention can not only oxidize the glucose molecule in the glucose solution but also can be fluorescent quenched by the H₂O₂ molecule formed by the oxidation of the glucose molecule in the glucose solution. Therefore, the worker can detect the glucose concentration of the glucose solution in a sensitive and specific way according to the resulting change in fluorescent intensity of the nanodot.

Moreover, by the use of the nanodot, the method for detecting glucose according to the present invention can be used to detect glucose concentration with high sensitivity and specificity.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A nanodot for detecting glucose concentration, comprising:
a silicon oxide core;
a self-assembled monolayer having a 3-glycidoxypropyl trimethoxysilane group, wherein the self-assembled monolayer joins the silicon oxide core by a covalent bond; and
a glucose oxidase particle joins the 3-glycidoxypropyl trimethoxysilane group of the self-assembled monolayer by a conjugated bond.

2. A method for detecting glucose concentration, comprising:
oxidizing a glucose molecule in a glucose solution by the glucose oxidase particle of the nanodot as claimed in claim 1, producing a hydrogen peroxide molecule;
fluorescent quenching the nanodot by the hydrogen peroxide molecule, resulting in a change in fluorescent intensity of the nanodot; and
detecting the change in fluorescent intensity of the nanodot.

3. The method for detecting glucose concentration as claimed in claim 2, wherein the change in fluorescent intensity of the nanodot is detected at 497 nm.
